(19) United States
(12) Patent Application Publication
(10) Pub. No.: US 2023/0071025 A1
(43) Pub. Date: Mar. 9, 2023

(54) GUIDANCE PROVISIONING FOR REMOTELY PROCTORED TESTS

(71) Applicant: EMED LABS, LLC, Miami, FL (US)

(72) Inventors: Nicholas Atkinson KRAMER, Wilton Manors, FL (US); Sam MILLER, Hollywood, FL (US)

(21) Appl. No.: 17/929,424

(22) Filed: Sep. 2, 2022

Related U.S. Application Data
(60) Provisional application No. 63/241,031, filed on Sep. 6, 2021.
(60) Provisional application No. 63/268,683, filed on Feb. 28, 2022.
(60) Provisional application No. 63/370,566, filed on Aug. 5, 2022.
(60) Provisional application No. 63/371,799, filed on Aug. 18, 2022.
(60) Provisional application No. 63/373,025, filed on Aug. 19, 2022.

(51) Int. Cl. A61B 5/00; G16H 10/20; G16H 10/60; G16H 20/00; G16H 80/00

(52) U.S. Cl. CPC A61B 5/0022; G16H 10/20; G16H 10/60; G16H 20/00; G16H 80/00; A61B 5/002

(57) ABSTRACT

This disclosure relates to systems and methods for remote diagnostic medical testing. Some embodiments relate to resource allocation. Some embodiments relate to dynamic resource allocation. In some embodiments, a method for remote diagnostic testing can include receiving a request to begin a testing session, selecting at least one guidance provision scheme from a plurality of guidance provision schemes, beginning the testing session using the selected at least one guidance provision scheme, receiving data indicative of one or more characteristics of the testing session, determining to modify the testing session for the user, and altering the testing session. In some embodiments a method can include determining, based on data indicative of the user's sentiment, one or more baseline scores associated with one or more emotion and detecting a change in the user sentiment during the testing session.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119(e) to U.S. Provisional Patent Application No. 63/241,031, titled “GUIDANCE PROVISIONING FOR REMOTELY PROCTORED TESTS,” filed Sep. 6, 2021, U.S. Provisional Patent Application No. 63/268,683, titled “SENTIMENT ANALYSIS FOR PROCTORED EXAMINATIONS,” filed Feb. 28, 2022, U.S. Provisional Patent Application No. 63/370,566, titled “SENTIMENT-DRIVEN AB TESTING AND TEST PATH DETERMINATION,” filed Aug. 5, 2022, U.S. Provisional Patent Application No. 63/371,799, titled “SYSTEMS METHODS AND DEVICES FOR SENTIMENT DRIVEN RESOURCE ALLOCATION FOR IMPROVED USER SATISFACTION,” filed Aug. 18, 2022, and to U.S. Provisional Patent Application No. 63/373,025, titled “ADAPTIVE TESTING,” filed Aug. 19, 2022, each of which are incorporated herein by reference.

### BACKGROUND

### Field

The present disclosure relates to remote medical diagnostic testing. More specifically, some embodiments relate to customized or adaptive test sessions using artificial intelligence proctoring.

### Description of the Related Art

The approaches described in this section are approaches that could be pursued, but not necessarily approaches that have been previously conceived or pursued. Thus, unless otherwise indicated, it should not be assumed that any of the material described in this section qualifies as prior art merely by virtue of its inclusion in this section.

The use of telehealth to deliver health care services has grown consistently over the last several decades and has experienced very rapid growth in the last several years. Telehealth can include the distribution of health-related services and information via electronic information and telecommunication technologies. Telehealth can allow for long-distance user and health provider contact, care, advice, reminders, education, intervention, monitoring, and admissions. Often, telehealth can involve the use of a user or patient's personal electronic device such as a smartphone, tablet, laptop, desktop computer, or other type of personal device. For example, the user or patient can interact with a remotely located medical care provider using live video and/or audio through the personal device.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

In some aspects, the techniques described herein relate to a method for remote diagnostic testing including: receiving, by a computer system from a user, a request to begin a testing session; selecting, by the computer system, at least one guidance provision scheme from a plurality of guidance provision schemes; beginning, by the computer system, the testing session using the selected at least one guidance provision scheme; receiving, by the computer system, data indicative of one or more characteristics of the testing session; determining, by the computer system based on the received data, to modify the testing session for the user; and in response to determining to modify the testing session for the user, altering, by the computer system, the testing session.

In some aspects, the techniques described herein relate to a method, wherein selecting the at least one guidance provision scheme is based on a user profile and a resource availability level.

In some aspects, the techniques described herein relate to a method, wherein the user profile includes at least one or a user experience level, demographic information, a number of times the user has taken a test, and information about previous positive or negative experiences of the user.

In some aspects, the techniques described herein relate to a method, wherein receiving data indicative of one or more characteristics of the testing session includes receiving data indicative of a user sentiment of the user, wherein determining to modify the testing session is based on the user sentiment.

In some aspects, the techniques described herein relate to a method, further including: determining, by the computer system based on the data indicative of the user sentiment, one or more baseline scores associated with one or more emotions; and detecting, by the computer system, a change in the user sentiment during the testing session.

In some aspects, the techniques described herein relate to a method, wherein determining to modify the testing session is based at least in part on detecting a change over a threshold amount of at least one of a negative emotion score, one or more baseline sentiment scores, or an overall sentiment score.

In some aspects, the techniques described herein relate to a method, further including triggering one or more interventions, the one of more interventions including at least one of placing the user in a high priority queue, allocating the user a high-value resource, or modifying the testing session.

In some aspects, the techniques described herein relate to a method, furthering including modifying a threshold amount based on a likelihood of a negative test outcome.

In some aspects, the techniques described herein relate to a method, further including: monitoring, by the computer system, user behavior, the user behavior including one or more of speech of the user, facial expressions of the user, and movements of the user, wherein the user data includes data indicative of the user sentiment.

In some aspects, the techniques described herein relate to a method, further including: receiving, by the computer system from the user, a request for an adjustment to the testing session; determining, by the computer system, that an adjustment to the testing session is available; and modifying the testing session in response to the user request for an adjustment to the testing session.

In some aspects, the techniques described herein relate to a method, further including: determining by the computer system, a type of adjustment requested by the user, wherein determining that an adjustment to the testing session is available includes determining that an adjustment corresponding to the type of adjustment requested by the user is available.

In some aspects, the techniques described herein relate to a system for remote diagnostic testing including: a non-transitory computer-readable medium with instructions encoded thereon; and one or more processors configured to execute the instructions to cause the system to: receive a request to begin a testing session from a user; select at least one guidance provision scheme from a plurality of guidance provision schemes; begin the testing session using the selected at least one guidance provision scheme; receive data indicative of one or more characteristics of the testing session; determine, based on the received data, to modify the testing session for the user; and in response to determining to modify the testing session for the user, alter the testing session.

In some aspects, the techniques described herein relate to a system, wherein selecting the at least one guidance provision scheme is based on a user profile and a resource availability level.

In some aspects, the techniques described herein relate to a system, wherein receiving data indicative of one or more characteristics of the testing session includes receiving data indicative of a user sentiment of the user, wherein determining to modify the testing session is based on the user sentiment.

In some aspects, the techniques described herein relate to a system, wherein the instructions, when executed by the one or more processors, further cause the system to: determine, based on the data indicative of the user sentiment, one or more baseline scores associated with one or more emotions; and detect a change in the user sentiment during the testing session.

In some aspects, the techniques described herein relate to a system, wherein determining to modify the testing session is based at least in part on detecting a change over a threshold amount of at least one of a negative emotion score, one or more baseline sentiment scores, or an overall sentiment score.

In some aspects, the techniques described herein relate to a system, wherein the instructions, when executed by the one or more processors, further cause the system to trigger one or more interventions, the one of more interventions including at least one of placing the user in a high priority queue, allocating the user a high-value resource, or modifying the testing session.

In some aspects, the techniques described herein relate to a system, wherein the instructions, when executed by the one or more processors, further cause the system to: monitor user behavior, the user behavior including one or more of speech of the user, facial expressions of the user, and movements of the user, wherein the user data includes data indicative of the user sentiment.

In some aspects, the techniques described herein relate to a system, wherein the instructions, when executed by the one or more processors, further cause the system to: receive, from the user, a request for an adjustment to the testing session; determine that an adjustment to the testing session is available; and modify the testing session in response to the user request for an adjustment to the testing session.

In some aspects, the techniques described herein relate to a system, wherein the instructions, when executed by the one or more processors, further cause the system to: determine a type of adjustment requested by the user, wherein determining that an adjustment to the testing session is available includes determining that an adjustment corresponding to the type of adjustment requested by the user is available.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

### BRIEF DESCRIPTION OF THE DRAWINGS

These and other features, aspects, and advantages of the present application are described with reference to drawings of certain embodiments, which are intended to illustrate, but not to limit, the present disclosure. It is to be understood that the attached drawings are for the purpose of illustrating concepts disclosed in the present application and may not be to scale.

FIG. 1A is a schematic diagram illustrating a proctored test system with a test user, user device, testing platform, network, proctors, and proctor computing devices.

FIG. 1B is a schematic diagram illustrating a system with logic for carrying out one or more guidance-provision scheme selection processes.

FIG. 2 is a schematic diagram illustrating a guidance-provision scheme selection process.

FIG. 3A is a plot that shows an example of user emotions for a testing session.

FIG. 3B is a plot that shows an example of user emotions during a testing session.

FIG. 4 is a flow chart of an example adaptive testing process according to some embodiments.

FIG. 5 shows an example outcome landscape according to some embodiments herein.

FIG. 6 shows an example process for measuring user experiences according to some embodiments.

FIG. 7 shows an example test flow according to some embodiments.

FIG. 8 is a block diagram depicting an embodiment of a computer hardware system configured to run software for implementing one or more embodiments disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As mentioned briefly above and as will now be explained in more detail, this application describes system, methods, and devices for guidance provisioning in proctored testing.

Remote or at-home health care testing and diagnostics can solve or alleviate some problems associated with in-person testing. For example, health insurance may not be required, travel to a testing site is avoided, and tests can be completed at a user's convenience. However, at-home testing introduces various additional logistical and technical issues, such as guaranteeing timely test delivery to a user's home, providing test delivery from a user to an appropriate lab, ensuring test verification and integrity, providing test result reporting to appropriate authorities and medical providers, guiding users through unfamiliar processes such as sample collection and/or processing, and connecting users with medical providers, who are sometimes needed to provide guidance and/or oversight of the testing procedures remotely.

While remote or at-home health care testing offers many benefits, there are several difficulties. Users may find a testing procedure confusing, frustrating, boring, and so forth. In some cases, users may be in a rush, upset, distracted, and so forth while taking a test. In some cases, users may become confused or frustrated during a test. Providing users with altered testing experiences can improve outcomes (e.g., user satisfaction, test result validity, and so forth), but it can be difficult to recognize when a test session should be altered to provide a better experience. Some embodiments described herein are directed to determining initial testing conditions for a user. Some embodiments described herein can be used to alter testing sessions during a testing procedure in order to improve the experience for the end user.

At-home or remote diagnostic testing can sometimes require users to complete complicated and/or unfamiliar steps. Often, these steps must be done correctly to ensure that test results are accurate. For example, collecting a sample, adding the sample to a test kit, mixing the sample with a reagent, and/or reading and interpreting results can present opportunities for a variety of pitfalls and errors that could render a test inaccurate. In some cases, an error may be recoverable (for example, if a user incorrectly reads a test result), while in other cases, a user may have to repeat a step or redo a test entirely in order to obtain a valid result. Thus, there is a need to provide clear guidance that even novice users can follow. However, many users will complete tests multiple times. Experienced users may not need in-depth instruction because they are already experienced with the testing procedure. Such users may instead prefer brief cues to remind them of how to complete the testing steps.

Proctored telehealth platforms or telehealth providers can have limited resources, especially high-quality or highly trained resources such as experienced proctors, managers, customer service representatives, and so forth. In some cases, platforms or providers may have limited computational capacity for deploying compute-intensive artificial intelligence (AI) or machine learning (ML) models. Telehealth providers can have resources of different quality levels due to financial or logistical constraints. The different quality levels can lead to tiered quality of care where a user's experience on the platform can be influenced by the quality of resources the telehealth provider allocates to the user. If telehealth providers randomly allocate resources or allocate resources on a first-come, first-served basis, users with a need for higher care can receive lower-quality resources, and high-quality resources can be allocated to users who do not need them.

A proctored telehealth platform with sentiment-driven resource allocation can allocate resources to one or more users in real-time or substantially real-time based at least in part on user needs. In some embodiments, a system can be a proctored telehealth platform. The system can use a sentiment engine to automatically allocate resources. In some embodiments, the sentiment engine can use artificial intelligence (AI) and/or machine learning (ML) to automatically allocate resources. The system can minimize a number of patients that have an unsatisfactory or insufficiently supported experience on the proctored telehealth platform. In some embodiments, the sentiment engine can use one or more of live or real-time sentiment signals, historical sentiment signals, user demographic data, user personality data, and so forth as discussed herein.

In some embodiments, the system can automatically determine if the user had or is having a positive or negative experience on the telehealth proctored platform. The system can use direct patient feedback (e.g., surveys), indirect patient feedback (e.g., sentiment analysis), a total test or visit time (e.g., comparing the test time or visit time to an expected or threshold time), and/or any other evidence of a user experience to automatically determine if the user had or is having a positive or negative experience. In some embodiments, the system can reduce negative experiences and minimize an average total test or visit time by decreasing an number of high-value resources allocated to users that do not require or derive much benefit from the high-value resources, and instead dynamically allocating appropriate resources based on a minimum quality level desired to complete a test or visit. In some embodiments, the system can dynamically allocate the appropriate resources before a test session or visit based on real-time or substantially real-time user information and/or a test type or procedure type associated with the test session or visit. In some embodiments, resources can be reallocated during testing sessions, as discussed in more detail below.

In some embodiments, the system can allocate resources based on a user profile. The user profile can include various information such as, for example, an experience level of the user, demographic information, previous positive or negative experiences of the user, etc. In some embodiments, the system can determine the experience level based on a number of times the user has taken a specific test or test type, how frequently the user has taken the specific test or test type, and so forth. In some embodiments, a user with a higher experience level (e.g., more experience and/or frequent test taking) may have a positive experience without using a highly trained or experience proctor. The user with the higher experience level can be self-sufficient with completing each step of a test and can correctly complete a test within a predetermined time without guidance from a proctor or with only limited guidance from the proctor. Thus, in some embodiments, the system can allocate new proctors, proctors with less experience, or proctors who have has less training to users with higher experience levels. In some embodiments, the system can allocate experienced proctors or highly trained proctors to users with a lower experience level (e.g., less or no test experience and/or infrequent test taking).

In some embodiments, the demographic information can include one or more of a user's age, sex, gender, geographic location, medical history, and any other personal information that can impact a user's tolerance or preference of proctor attributes. In some embodiments, the system can use principles or patterns automatically determined by the sentiment engine to allocate resources to the user. The principles or patterns can be generalities or patterns based on aggregated data (e.g., surveys, test times, etc.). In some embodiments, the principles or patterns can be a function of the demographic information. For example, one demographic may be less tolerant or may not prefer proctors that speak slowly, or users from certain geographic locations may have a preference for physicians over physician's assistants. In some embodiments, a sentiment engine can automatically and dynamically update the principles or patterns. The sentiment engine can analyze interaction data such as speech patterns, tone, and/or body language of the proctor or the user by analyzing video and audio data from a test session. In some embodiments, the sentiment engine can automatically detect positive or negative interaction data and update the patterns or principles based on the positive or negative interaction data. For example, a user may be from the Midwest, and the sentiment engine can automatically detect that the user is irritated by slow speech of a proctor. The system can dynamically update principles or patterns associated with users from the Midwest based on the user's experience. In some embodiments, the principles or patterns can be updated based on a threshold number of users who fit within a particular demographic expressing similar sentiment in similar situations.

In some embodiments, the system can determine previous positive or negative experiences of the user based on, for example, a user rating of a telehealth session, automatic detection of a positive or negative experience by the sentiment engine, whether a user contacted customer service during or after a telehealth session, and/or any other evidence of a positive or negative experience. The system can allocate resources to the user based on factors that caused a user to previously have a positive or negative experience. The system can automatically and/or dynamically determine correlations between proctor attributes and previous positive or negative experiences to determine proper proctor attributes for the user. For example, if the user previously has a negative experience with a poorly rated or new proctor, the system can allocate a highly trained proctor to the user. In some embodiments, the system can automatically update the user profile after each telehealth session. In some embodiments, the system can assign the user profile with a premium or priority status. The premium or priority status can be assigned after each telehealth session or when the user uses the proctored telehealth platform a next time. In some embodiments, the premium or priority status can be assigned after the user has a negative experience, and the premium or priority status can be assigned until the user has a positive experience.

In some embodiments, the sentiment engine can determine a user experience threshold. The user experience threshold can indicate a minimum proctor experience the sentiment engine determines as necessary or preferable for a specific user to have a positive experience. The system can allocate resources based on the user experience threshold. The system can allocate resources to the user that are at or above the user experience threshold, or available resources that are the closest to the user experience threshold.

In some embodiments, the user may have a negative experience based on a current or previous wait time. In some embodiments, the system can allocate resources or enact equalizing measures to reduce the user wait time the next time the user uses the telehealth platform. For example, the system may move the user up in a queue ahead of other users, provide discounts to the user for previous or future telehealth sessions, automatically send apology gifts, gift cards, etc. to the user, and/or otherwise prioritize the user to improve the user's experience.

In some embodiments, the system can automatically allocate resources in real-time or substantially real-time to perform real-time matchmaking. In some embodiments, the system can automatically update or change sentiment observations associated with a user. The sentiment engine can automatically determine a baseline sentiment score associated with one or more emotions (e.g., anger, aggravation, confusion, impatience satisfaction, etc.) of the user. In some embodiments, the system can aggregate multiple baseline sentiment scores associated with the one or more emotions into an overall sentiment score. In some embodiments, the overall sentiment score can represent how positive or negative a user's overall experience is for a proctored telehealth session or for multiple proctored telehealth sessions.

In some embodiments, the sentiment engine can update the baseline sentiment scores and the overall sentiment score based on each user interaction with the proctored telehealth platform or dynamically throughout each user interaction. In some embodiments, the sentiment engine can detect sharp changes (e.g., sudden large increase or decrease) in the baseline sentiment scores and/or the overall sentiment score. In some embodiments, the sentiment engine can prioritize recognition of decreases in the baseline sentiment scores and/or the overall score (e.g., a decrease in positive emotion or increase in negative emotion). In some embodiments, the sentiment engine can dynamically determine a negative emotion score throughout a user interaction. In some embodiments, the negative emotion score can be a cumulative score of each negative emotion of the user throughout the user interaction. In some embodiments, the negative emotion score can be associated with a change of the baseline sentiment scores associated with one or more negative emotions, a change of the baseline sentiment scores associated with one or more positive emotions, and/or a decrease of the overall sentiment score.

In some embodiments, if the negative emotion score exceeds a predetermined threshold, and/or the sentiment engine detects a sharp change in the negative emotion score, one or more baseline sentiment scores and/or the overall sentiment score, the system can trigger one or more interventions. In some embodiments, the one or more interventions can include placing the user in a high priority queue, allocating the user a high-value resource, or any other intervention to address the negative emotion of the user. In some embodiments, the one or more interventions may be based on a specific negative emotion of the user. For example, if the sentiment engine detects a sharp increase in a baseline sentiment score associated with impatience, or the negative emotion score associated with impatience increases above the predetermined threshold, the system can place the user in a high priority queue to reduce the user's wait time and thereby reduce the negative emotion. In another example, if the sentiment engine determines the negative emotion score associated with confusion increases above the predetermined threshold, the system can allocate the user a proctor with a high emotional intelligence rating to address the user's confusion. In some embodiments, the sentiment engine can determine an emotional intelligence rating of a proctor based on an average change in the baseline sentiment score and/or overall sentiment score of every user the proctor interacts with on the proctored telehealth platform. For example, a proctor can have a high emotional intelligence rating if the sentiment engine detects that a proctor on average makes users less confused, less frustrated, and so forth, or if the proctor is more effective at providing information to users than other proctors (e.g., 20% more effective, 30% more effective, 40% more effective, etc.).

In some embodiments, the baseline sentiment score can include an average mood of the user. The average mood of the user can include scores from one or more prior tests and/or scores obtained prior to a user interaction, such as a prescreening process. The system can use the average mood of the user to account for various temperaments of each user.

In some embodiments, if the user interaction can have a negative outcome, the system can reduce the predetermined threshold level to compensate for the possibility of the negative outcome. The negative outcome can include a positive test result, poor prognosis, requirement of a follow up to confirm or receive results, or any other negative outcome typically associated with diagnosis or health care. By reducing the predetermined threshold, the system can provide extra care or intervention to users that are likely to feel negative emotions not associated with the testing process of the telehealth platform. The system can reduce a negative experience of a user by triggering one or more interventions at a lower threshold. In some embodiments, the system can automatically limit the one or more interventions to high-quality resources or proctors. For example, if the system determines a negative outcome is likely, the system can allocate a proctor that the system determines has a high bedside manner rating, because proctors with high bedside manner can be well suited to interact with a user that received a negative outcome.

In some embodiments, resources can include proctors, computer-based resources, for example highly trained AI models or general AI models, healthcare providers, such as physicians, physician's assistant or nurses, or any other telehealth resource.

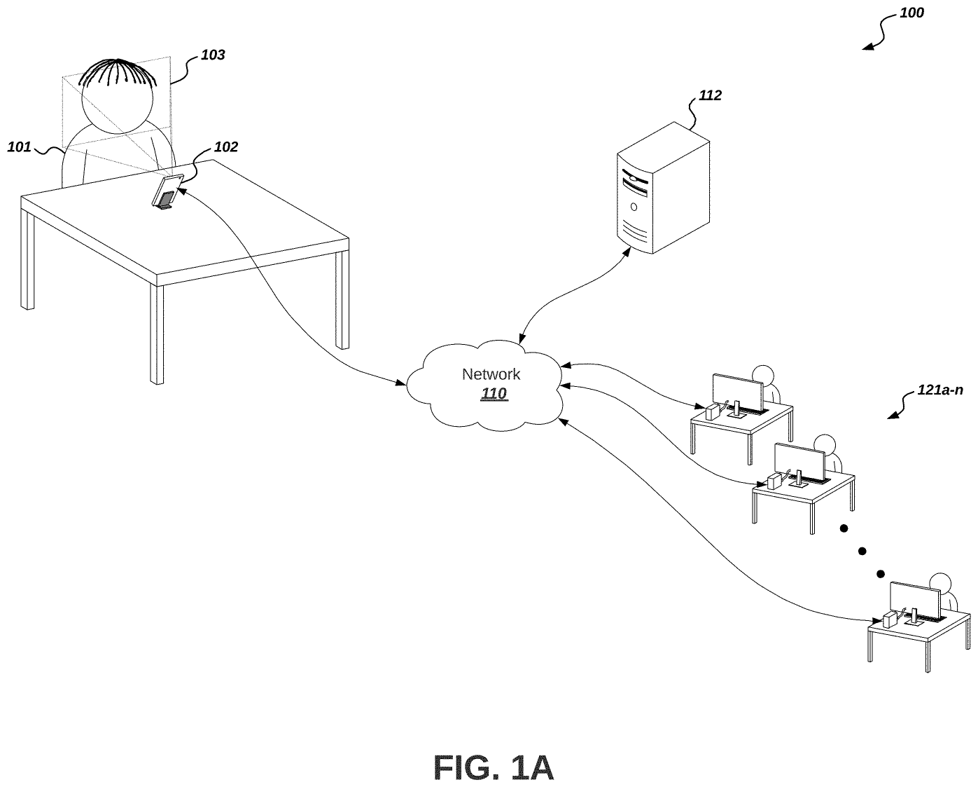

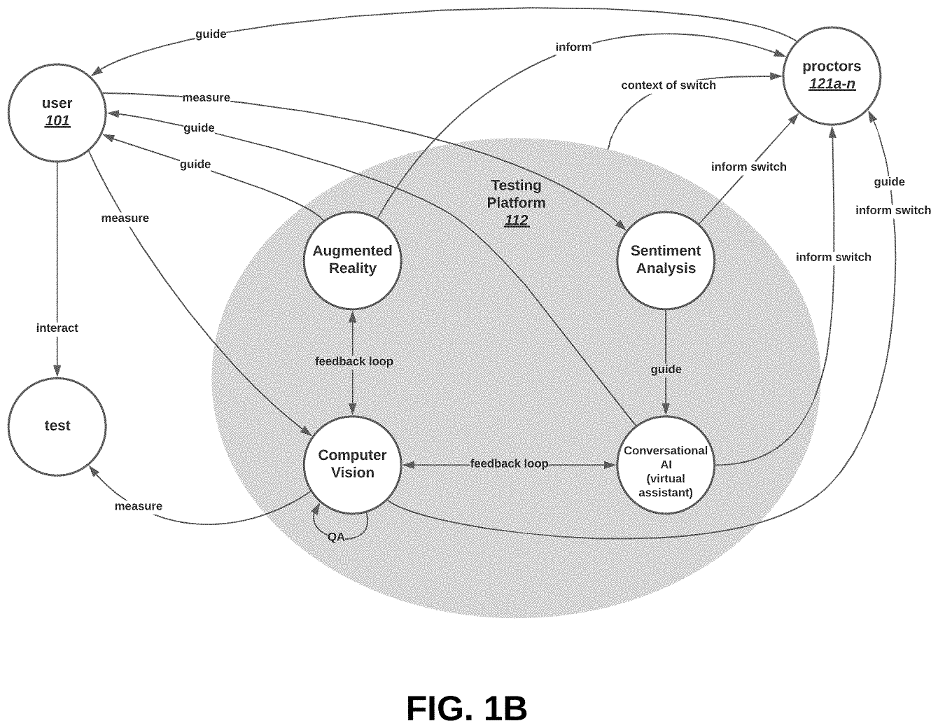

FIG. 1A provides a schematic diagram illustrating a proctored test system. As shown in FIG. 1A, a user 101 may undergo a remotely proctored test (which can be, for example, a health test or diagnostic) using a user device 102, which may be a smartphone, tablet, computer, etc. The user device 102 can be equipped with a camera having a field of view 103. The user 101 may perform one or more steps of the remotely proctored test within the field of view 103 of the camera, such that such steps can be monitored by a proctor (e.g., a proctor selected from proctors 121a-n). In some embodiments, the user 101 may be monitored by a live proctor. In some embodiments, the user 101 may be monitored by an artificial intelligence (AI) proctor. In some embodiments, a human proctor or an AI proctor may monitor the user live (e.g., in substantially real-time). In some embodiments, the proctor may access a recording of the user, such that monitoring does not occur in real-time.

With continued reference to FIG. 1A, a plurality of proctors 121a-n may monitor and guide users on the testing platform 112 over a network 110. In some embodiments, each proctor 121a-n may monitor more than one user simultaneously. For example, in some embodiments, a single proctor 121a-n may monitor one, two, three, four, five, six, seven, eight, nine, ten, fifteen, twenty, twenty-five, fifty or more users at a time. In some embodiments, proctors may monitor the user 101 during all steps in the administration of the proctored test. In some embodiments, the user 101 may interact with the same proctor over the course of a proctored test. In some embodiments, proctors may monitor the user 101 during certain steps in the administration of the proctored test. In some embodiments, the user 101 may interact with different proctors over the course of a proctored test (e.g., at different stages or steps of the session). Even so, in some embodiments, there may not be enough proctors available for all users, especially in instances of increased user demand.

FIG. 1B shows a conceptual framework associated with a system 100 in which logic for carrying out one or more guidance-provision scheme selection processes is employed at the testing platform 112. In some embodiments, the testing platform 112 may have capabilities for sentiment analysis, augmented reality, computer vision, and/or conversational artificial intelligence (e.g., a virtual assistant, chatbot, etc.), among others.

For example, an augmented reality (AR) module of the testing platform 112 can provide AR guidance to the user. For example, AR guidance illustrating or providing information about a testing step can be overlaid onto the display of the user device 102. Additionally or alternatively, the AR module can also provide AR guidance which provides the proctors 121a-n with various types of information during the test. Such AR guidance can be overlaid onto a display that the proctors 121a-n use to administer the proctored testing session and/or monitor the user during the same.

A sentiment analysis module of the testing platform 112 can be configured to measure a sentiment of the user 101. For example, the sentiment analysis module can analyze an image or video of the user, an audio recording of the user, data input by the user (e.g., text-based data input via the user device), or other types of information to measure, determine, or estimate a sentiment of the user. In some embodiments, the sentiment analysis module can be configured to detect negative sentiments (e.g., frustration, confusion, annoyance, etc.) so that the testing platform 112 can take steps to remedy the situation in order to provide a more positive user experience. Accordingly, in some embodiments, user sentiments determined by the sentiment analysis module can be sent to the proctors 121a-n or to a conversational AI (virtual assistant), who can take appropriate remedial action.

The conversational AI can be a module configured to converse with the user (e.g., through text (such as a chatbot) or voice without requiring the use of a live proctor 121a-n). This can be advantageous as the conversational AI can be provided on-demand, even when a live proctor 121a-n is not available. This can provide immediate assistance to the user. In the event that the conversational AI is determined to be unable to address a user issue, the user 101 can be passed from the conversational AI to a live proctor 121a-n.

The testing platform 112 can also include a computer vision module. The computer vision module can be configured to analyze and measure information provided from the camera of the user device.

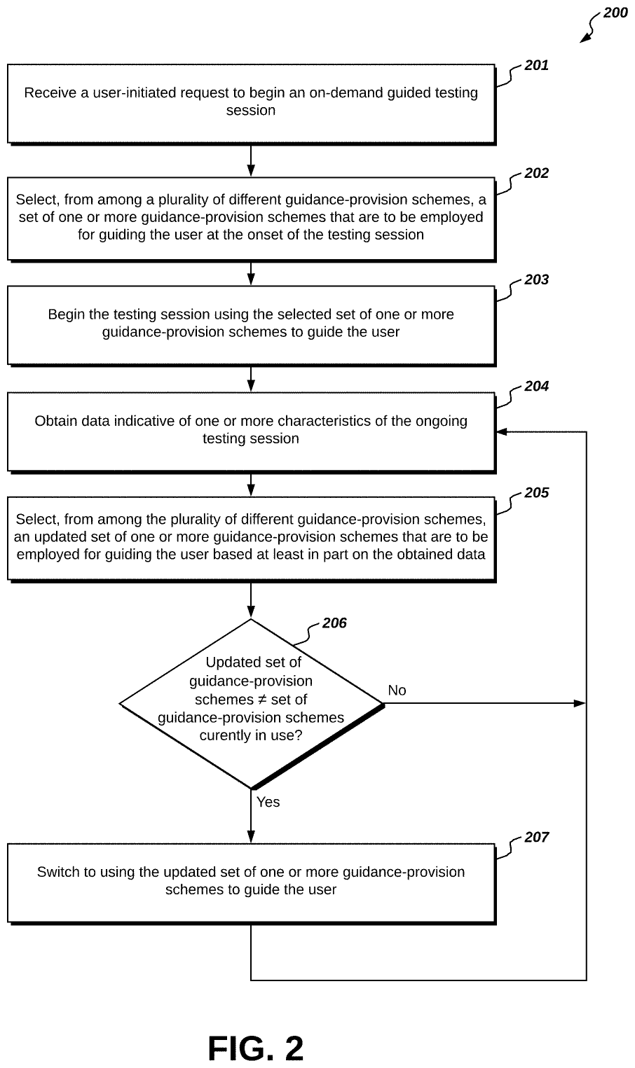

FIG. 2 illustrates an example flow 200 through a remotely proctored test with guidance-provision scheme selection. At 201, the testing platform receives a user-initiated request to begin a guided testing session. At 202, the testing platform selects one or more guidance-provision schemes that are to be employed for guiding the user at the onset of the testing session. In some embodiments, a plurality of different guidance-provision schemes may include, for example, an augmented reality-based guidance-provision scheme, a virtual assistant-based guidance-provision scheme, or a proctor-based guidance-provision scheme. In some embodiments, the selection of one of more guidance-provision schemes may be based on information such as, for example, user preferences, user profile information, traffic volume on the testing platform, or proctor availability, among others. In some embodiments, when a proctor-based guidance-provision scheme is selected for a user, the platform may perform one or more operations to match the user with a suitable proctor. In some embodiments, selecting a guidance-provision scheme can include selecting one or more parameters such as, for example, a speaking speed (e.g., fast, or slow), an instruction level (e.g., brief guidance, detailed instructions, etc.), and so forth. In some embodiments, the guidance-provision scheme or parts of the guidance provision scheme can be altered during the testing session as discussed in more detail below. In some embodiments, the guidance provision scheme can be altered based on explicit user requests, user behavior, etc. In some embodiments, different types of guidance may be available at different steps in a testing process as described herein.

As further illustrated in FIG. 2, at 203, the testing platform begins the testing session using a selected set of one or more guidance-provision schemes to guide the user. At 203, the testing platform obtains data indicative of the ongoing testing session. In some embodiments, the data obtained may include, for example, one or more of: data indicative of whether the user has reached a step in the testing procedure that requires proctor guidance or supervision (such as, for example, steps that are required by applicable regulations to be observed by a proctor); data indicative of a user's current sentiment such as, for example, gesturing, cursing, sighs, groans, movement, vocal tone, vocal volume, speech frequency, speech speed, facial expression, or a combination thereof; data indicative of the amount of difficulty the user may be experiencing in performing steps of the testing procedure; data indicative of whether the user may be attempting to engage in fraudulent test-taking practices (such as, for example, moving out of the field of view); data indicative of whether one or more technical failures have occurred during the testing session; data indicative of whether artificial intelligence-based functions can be considered to be reliable under current testing conditions (such as, for example, network connection or lighting conditions); data indicative of the volume of users accessing the testing platform; data indicative of the availability of proctors. In some embodiments, such obtained data may include data obtained by applying one or more machine learning algorithms such as, for example, computer vision, sentiment analysis, or others to input data received from the user's device such as, for example, audio or video.

In some embodiments, the testing platform at 205 selects, from among the plurality of different guidance-provision schemes, an updated set of one or more guidance-provision schemes that are to be employed for guiding the user based at least in part on the data obtained at 204. In some embodiments, the data obtained at 204 may indicate that the user is frustrated, annoyed, or otherwise unhappy, and the testing platform may select a proctor-based guidance provisioning scheme at 205 for the purpose of providing remediation. In some embodiments, the data obtained at 204 may indicate that the user is confused or experiencing difficulty, and the testing platform may select an augmented reality-based guidance-provision scheme, a proctor-based guidance scheme, or both, for purposes of aiding the user. In some embodiments, the data obtained at 204 may indicate that there is a surplus of available proctors, and the testing platform may select a proctor-based guidance-provision scheme for purposes of increasing efficiency. In some embodiments, the data obtained at 204 may indicate that the testing conditions, such as for example lighting, are inadequate for augmented reality or artificial intelligence-based guidance, and the testing platform may select a proctor-based guidance scheme.

In some embodiments, the testing platform determines at 206 whether the updated set of one or more guidance-provision schemes selected at 205 differs from the previously-selected set of one or more guidance-provision schemes that is currently being used in the testing session. In some embodiments, if the updated set of one or more guidance-provision schemes selected at 205 includes a proctor-based guidance-provision scheme and the previously-selected set of one or more guidance-provision schemes that is currently being used in the testing session does not include such a proctor-based guidance-provision scheme, the testing platform may further perform one or more operations to match the user with a suitable proctor.

In some embodiments, in response to determining that the updated set of one or more guidance-provision schemes differs from the set of one or more guidance-provision schemes that is currently in use, the testing platform switches at 207 to the updated set of one or more guidance-provision schemes to guide the user.

In some embodiments, facial recognition techniques can be used to estimate a range of user emotions over time. In some embodiments, key emotional indicators can be aggregated to determine an overall user sentiment or net positivity score. In some embodiments, customer satisfaction can be automatically tracking and tagged throughout testing stages. As discussed above, user sentiment can be used to redirect or alter a user's testing experience. User sentiment can also be used in other ways, for example for testing features or test flow paths (e.g., A/B testing), to develop business insights, etc. In some embodiments, user sentiment analysis can be used to identify proctor training needs. For example, sentiment analysis may indicate a general need for proctor training on particular steps, procedures, and so forth. In some embodiments, sentiment analysis may indicate that a particular proctor should be provided with additional training, for example if users tend to have a more negative sentiment at particular steps or during particular procedures with the proctor as compared to users who interact with other proctors.

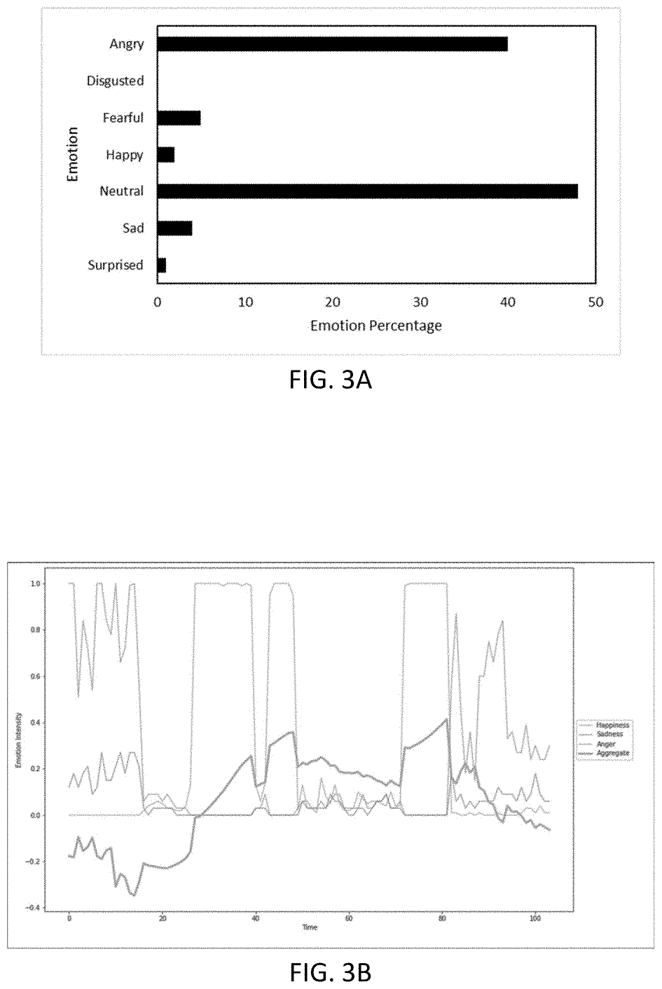

FIG. 3A is a plot that shows an example of user emotions for a testing session. As shown in FIG. 3A, in some embodiments, the user's emotions can be normalized or otherwise processed so that, for example, a user's predominant emotional expression at any given time can be considered, so that the percentages add to 100%, although other approaches are possible, and the total does not necessarily have to be 100%. FIG. 3B is a plot that shows an example of user emotions during a testing session. As shown in FIG. 3B, a user can express a variety of emotions during a testing session, and in some cases can express more than one emotion at once. For example, a user may be both sad and angry at the same time. In some embodiments, a user can have an overall or aggregate emotional intensity. In some embodiments, the emotional intensity can be positive or negative. For example, negative values may indicate the negative emotions such as anger or fear predominate, while positive values may indicate that the user is happy.

In some embodiments, as discussed briefly above, real-time signals such as gesturing, cursing, sighs, groans, movement, vocal tone, vocal volume, speech frequency, speech speed, facial expressions, voice sentiment, text sentiment (e.g., as determined by natural language processing algorithms), and so forth can be used to trigger escalation. In some embodiments, a frustration signal for the user can be determined. In some embodiments, the user's frustration signal can be compared to historical signals to determine if escalation should occur. In some embodiments, a user who is becoming frustrated may be escalated through various levels of care, preferably before the customer reaches undesirable levels of frustration. In some embodiments the various levels of care can include, for example, AI proctoring, multiplexed proctoring (e.g., the user works with a live proctor, but the proctor is not dedicated to the user), dedicated proctoring, proctoring by a highly trained proctor, escalation to customer service, escalation to a manager, and so forth. In some embodiments, baselines can be established for repeat users, for example by asking the user to rate their experience at the end of a testing session. In some embodiments, historical baseline data can be used to determine a predicted experience for a user. Advantageously, such approaches can enable a telemedicine provider to use abundant resources (e.g., AI, proctors with less training or experience, and so forth) whenever possible, but can escalate users to more expensive or less available resources (e.g., customer service, highly trained proctors, etc.) before the user becomes too frustrated, in contrast to an approach where users are only escalated after becoming frustrated.

As mentioned above, in some embodiments a testing platform 112 can predict an experience for a user. A predicted experience can be based on, for example, a dataset of other all other users and can include information such as session rating, demographic data, wait ties, proctor ratings, session length, test repeats, time of day, etc. In some embodiments, rolling windows can be used so that the most relevant data is prioritized. In some embodiments, outside factors can be considered such as, for example, whether there has been a recent uptick in disease cases, whether there is an emerging strain with worse symptoms or greater ability to spread, whether there is an upcoming holiday when people are more likely to travel or gather, and so forth. In some embodiments, for first time users, a system can compare predicted experiences to measured experiences to determine relevant variables such as the user's emotional range, best fit parameters, baseline parameters, and so forth. In some embodiments, for repeat users, the system can be configured to refine prior data and/or calculations for the user, etc. In some embodiments, the system can use periodic signals to predict what the user's sentiment will be in response to upcoming events. For example, prior data may indicate that a user becomes annoyed in certain parts of a testing session or once the testing session goes past a certain length. Prior data may indicate that a user becomes annoyed when asked to wait (e.g., to wait for a proctor, to wait for a test result, etc.), when asked to respond to risk questionnaire inquiries, when presented with an AI proctor, when presented with a human proctor, etc. In some embodiments, the sentiment analysis engine can be used for business intelligence.

In some embodiments, proctors may be able to provide feedback for a user. For example, a proctor may flag that a user was angry, annoyed, upset, and so forth.

In some embodiments, a system can be configured to perform a causal analysis of signals such as vocal tone, vocal volume, speech frequency, sentiment (e.g., as determined using natural language processing algorithms), facial expressions, movement, gesturing, cursing, sighs, groans, and so forth. In some embodiments, such information can be combined into a happiness signal. In some embodiments, the happiness signal can be used to determine the quality of the experience for the user at each step, across steps, and so forth. The happiness signal can be used to determine quality at particular steps or across multiple steps across many trials. In some embodiments, each testing procedure can be annotated with a happiness signal to assess which testing steps provide the best user experience. In some embodiments, annotations can be used to indicate steps that can be targeted for improvement, to identify high quality procedures that can be utilized elsewhere (e.g., at other steps in the same testing procedure or in different testing procedures, for example for a different type of test). In some embodiments, annotations can be used for comparative A/B testing for a step or procedure. Such approaches can provide granular information that be used for prioritizing areas for improvement, determining if new or changed procedures are working or better than previous procedures, and so forth. In some embodiments, sentiment comparison between procedures, between steps, between users, between types of users, and so forth can be automated.

It can be important to optimize a testing procedure so that it takes as little time as possible for a user to complete and does not burden the user with too much instruction or guidance, which can cause some users, especially experienced users, to become frustrated with the procedure. However, if too little information is provided, users may become confused, stuck, perform steps incorrectly, and so forth. Thus, in some embodiments, the testing platform 112 can be configured to detect when a user may be confused. In some embodiments, real-time signals can indicate that a user is taking too long at a given step. Various indications such as time taken, long vocal pauses, facial expressions, cursing, stopped action, hesitation, staring at a step, taking an incorrect action, and so forth can be used to determine a confusion signal. In some embodiments, if a confusion signal indicates that the user is struggling to complete a step, the testing platform 112 can be configured to take one or more actions in response. For example, the testing platform 112 can be configured to present the user with a longer or more detailed explanation of what to do, can provide the user with a tutorial, can slow down an AI voice, can direct the user to a live proctor, and so forth.

As discussed above, in some embodiments, a remote test may be performed with the aid of an artificial intelligence (AI) proctor. A testing platform can use AI proctors that guide users through an at-home diagnostic test. The AI proctors can be configured to watch and/or listen for input from the user. The AI proctors can be configured to interpret user input. In some embodiments, the user input can be explicit. In some embodiments, the user input can be implicit or non-verbal cues. In some embodiments, the input may be, for example, verbal cues (for example, specific key words, speech patterns, pauses, intonation, etc.), body language (for example, hand movements, posture, etc.), and/or other nonverbal cues, such as facial expressions, eye gaze, and so forth. In some embodiments, user inputs can be used by the AI proctor to adjust the testing procedure by altering the guidance provided to the user. The adjustments can be directed to providing a minimum amount of instruction for each user to perform each step of the test correctly. In some embodiments, the AI proctor may be configured to prioritize accuracy. In some embodiments, the AI proctor may be configured to prioritize a positive user experience, for example as determined by sentiment analysis, user surveys, etc.

In some embodiments, an AI proctor can detect that a user is taking too long to complete a step in the testing process. For example, the time spent by the user at a step can be compared to aggregated data of prior test takers, compared to a predetermined threshold time, etc. In some embodiments, the AI proctor may determine that the user is taking too long if, for example, the time spent by the user is in the top 5th percentile of time taken by test takers. In some embodiments, the AI proctor may be configured to provide a reminder instruction indicating what the user is supposed to do at the step.

In some embodiments, a user may not complete a step after being provided with reminder instructions, or the user may complete a step incorrectly. In some embodiments, the AI proctor may be configured to provide a longer and/or more detailed explanation of the step. For example, in a typical instruction, a user may be provided with an instruction such as, “Place three drops in hole,” while a more detailed instruction could be, for example, “Find the provided dropper container and remove the cap; position the dropper container over the top hole located on the right side of the test card; and dispense three drops into the hole.”

In some embodiments, multiple versions of instructions may exist for each step. The instructions can range from very short to very detailed. In some embodiments, the AI proctor may begin with the shortest version of the instructions and may escalate to longer instructions if user input indicates that the user is confused, misunderstanding, or otherwise struggling to complete a step. In some embodiments, the AI proctor may begin with a medium level of instruction detail or even a long level of instruction detail. In some embodiments, the beginning level of instruction detail can depend on, for example, whether the user is experienced with the test and/or the testing platform. In some embodiments, the level of instruction detail can be varied throughout the testing session based on whether the user appears to need more or less instruction.

In some embodiments, the AI proctor may identify key words that can help the user complete sub-steps of a test correctly. For example, in the scenario outlined above, an AI proctor may determine that a user has located the bottle and has removed the cap but has not deposited the three drops into the hole. The AI proctor may provide specific instruction to help the user identify the location of the hole within the test card where the drops should be deposited. In such a scenario, key words may include, for example, “test card,” “right side,” and/or other words associated with the location of the hole.

In some embodiments, the AI proctor may detect long vocal pauses after asking the user a question. Such a pause may indicate that the user did not hear and/or did not understand the question. The AI proctor may, in response to the pause, provide additional prompting to the user. For example, the AI proctor may ask if the user would like to hear the instruction again, would like a different explanation, would prefer a different language, would like to watch an instructional video, and so forth. In some embodiments, the AI proctor may present such inquiries using audio, text, graphics, and/or augmented reality.

In some embodiments, a user may be distracted. The AI proctor may detect that the user is distracted, for example based on eye contact, gaze direction, speaking, and so forth. In some embodiments, the testing platform may pause and wait for the user's attention to return to the test before proceeding with further instruction.

In some embodiments, a user's hand motions or other gestures may suggest that a user is experienced. For example, if a user reaches for a test kit object that is needed in the next step of the test, the AI proctor may determine that the user is experienced. The AI proctor may provide the user with less guidance. In some embodiments, the AI proctor may change the testing experienced to an experienced user mode that uses an abbreviated set of instructions.

In some embodiments, the AI proctor may detect frustration on the part of the user. For example, the user may utter negative statements, shout, or otherwise indicate that they are frustrated. The AI proctor may interpret such behaviors as indicating that the user is confused about or otherwise struggling with the test procedure. In response, the AI proctor may provide the user with more detailed instructions. In some embodiments, the AI proctor can determine that the user is frustrated because they feel that the testing procedure is too involved, the instructions are too long, and so forth. For example, if a user is being provided with relatively long instructions and appears frustrated but is completing steps correctly, the AI proctor may adjust the testing experience to provide less instruction to the user.

In some embodiments, a user may continue to express frustration after a level of instruction is adjusted. In some embodiments, the testing platform may direct the user to a human proctor. In some embodiments, directing the user to a human proctor can be based on the user's request for a human proctor or for further assistance. In some embodiments, the system can automatically direct the user to a human proctor.

In some embodiments, the AI proctor may adjust the testing experience based on a user's explicit request to speed up or slow down the speed of the procedure, to provide more or less detail, and so forth.

In some embodiments, the speaking speed of the AI proctor can be adjusted. In some embodiments, a user who is encountering little or no difficulty with a testing procedure may automatically have the speaking speed of the AI proctor increased. In some embodiments, a user who has encountered difficulty with the testing procedure may automatically have the AI proctor speed decreased.

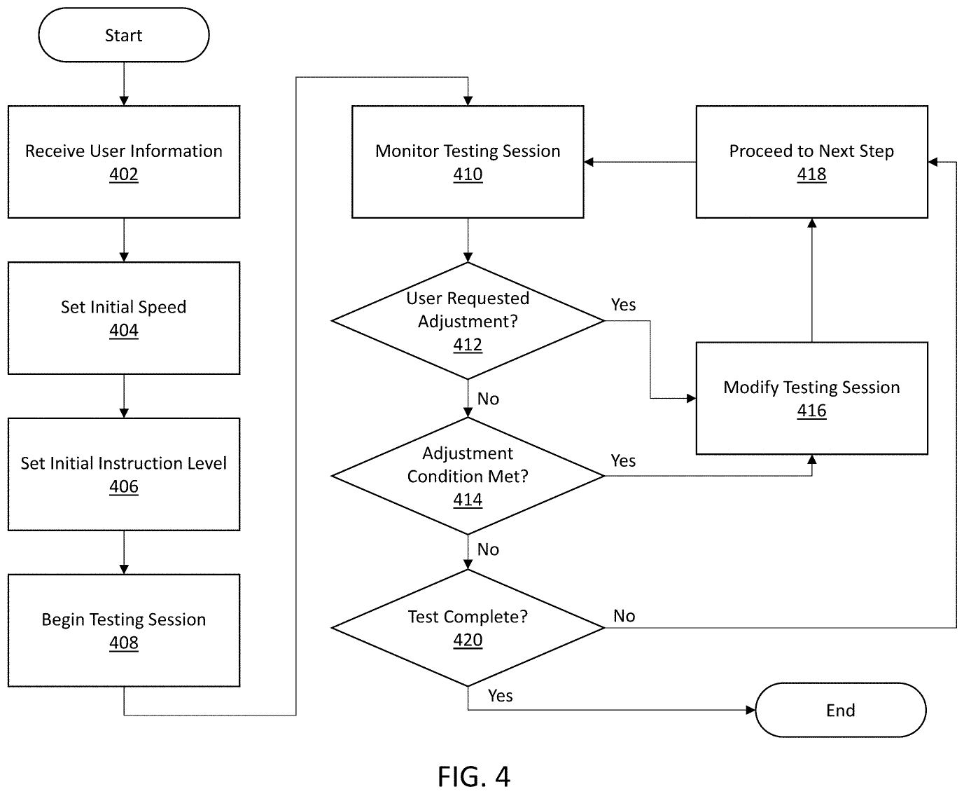

FIG. 4 is a flow chart of an example testing process. In some embodiments, a testing process can include more steps, fewer steps, and/or steps can be performed in an order different than is shown in FIG. 4. The process depicted in FIG. 4 can be carried out on a computer system.

At block 402, the system can receive user information. For example, a user can log in to a testing platform and can provide various information such as contact information, demographic information, medical information, and so forth. In some embodiments, the platform may record the user's activity and thus may have knowledge of whether the user is experienced with the platform, experienced with the particular diagnostic test being taken, and so forth.

At blocks 404 and 406, respectively, the system can set an initial speed level of an AI proctor (e.g., the speed at which the proctor speaks to the user) and can set an initial instruction level for the user. In some embodiments, the initial speed level and/or initial instruction level can be determined based at least in part on the user information received at block 402. For example, if the user is experienced, the initial speed level may be relatively fast and/or the initial instruction level may be relatively low, as an experienced user is likely to need less detailed instructions than a user who is new to the test, the testing platform, or both.

At block 408, the system can begin the testing session. At block 410, the system can monitor the testing session, for example by monitoring gestures, facial expressions, vocal expressions, time taken on a step, and so forth, as described in more detail above. At block 412, the system can detect if the user has explicitly requested an alteration to the test, such as speeding up or slowing down or providing more or less instruction. At block 414, the system can determine if a test alteration condition has been met. For example, a test alteration condition can include the detection of user frustration and/or confusion, distraction, boredom, and so forth, as explained in more detail above. While block 412 detects explicit user requests, block 414 uses artificial intelligence and/or machine learning techniques to automatically recognize conditions that indicate that the user can benefit from an alteration to the testing session. If, at block 412 the user requests an alteration or at block 414 the system detects that an alteration may be beneficial (or both), the system can, at block 416, adjust the speed, instruction level, or both of the testing session. At block 418, the system can proceed to the next step of the testing procedure.

At block 420, the system can determine if the testing session (or an AI-proctored portion of the testing session) is complete. If the session is not complete, the system can, at block 418, continue the testing session. If the testing session (or portion thereof) is complete, the system can end the testing session (or portion thereof).

As discussed above, remote, or at-home testing can offer many benefits. However, it can be difficult to determine an optimal testing approach. Different approaches may be advantageous for different users, for different tests, and so forth. Some embodiments herein can provide improved testing experiences for users, more efficient resource usage, and so forth. Preferably, testing procedures can be designed to provide a positive user experience and to remove, rewrite, or other improve testing steps that users find difficult, frustrating, and so forth.

There are many design decisions that can be made when developing an at-home or remote test, which may in some cases be a proctored test. For example, instructions can be thorough or concise, the instructions and/or proctor can take a direct tone or a more conversational tone, the test can be self-guided (and optionally including monitoring by a proctor, which may occur in real-time, substantially real-time, or after a test session is complete) or guided by a proctor, the proctor can be highly trained or relatively new, and so forth. In some cases, a test process can be modified to use an entirely different script and/or procedure. Conventionally, testing providers can gather user feedback on sessions in the form of a star rating, numeric rating, or otherwise, and A/B testing can be performed by providing different users with different testing experiences. However, user feedback often lacks granularity (for example, users may tend to rate a testing session either one star or five stars, with little in between). Moreover, user feedback can be influenced by outside factors, such as whether the user tested positive or negative, whether the user's own internet connection was stable, and so forth. Thus, traditional A/B testing can be difficult and give testing providers limited guidance for designing tests. Moreover, traditional feedback mechanisms lack the ability to gauge mid-test whether a user is happy with the experience or would be happier with a different experience, such as a different script flow that offers more or less guidance.

In some embodiments, a sentiment engine can intake a variety of indicator streams, such as facial expressions, language (for example, using language processing), and so forth, which may be indicative of a user's experience with the testing process. The sentiment engine can have a variety of outputs that indicate, for example, whether the user was confused, frustrated, enjoyed the overall test, and so forth. In some embodiments, different weights can be applied within a model to synthesize the information received from the various indicator streams. In some embodiments, the user's sentiment can be measured at different steps in the testing process, which may allow individual steps to be assessed.

In some embodiments, a test analysis platform can include a directed graph of all possible paths through a test. Each node can represent a state that a user is in, and each edge can represent a decision point. The branching from nodes can include, for example, long vs. short scripts, A/B test steps that are to be compared for test development, various levels of care, success and failure criteria, test to treat (for example, directing a user to treatment), and so forth. In some embodiments, the graph can contain every possible traversal of the testing procedure with all sets of outcomes. In some embodiments, the decision points can be defined by the testing platform provider (e.g., whether to escalate a user, provide a risk management questionnaire, direct the user to a different type of experience, etc.) and/or by the test itself (e.g., whether the user tests positive or negative). The graph can be referred to as an outcome landscape.

In some embodiments, many users (e.g., hundreds, thousands, tens of thousands, hundreds of thousands, or even more) can flow through the outcome landscape. In some embodiments, the sentiment engine can be run on all users or a subset of users to analyze their testing sessions. For example, the sentiment engine can be run on a random selection of users, a particular subset of users (e.g., a particular region, age range, sex, gender, education level, etc.). The results of the sentiment engine analysis can be used to develop a sophisticated understanding of the expected emotional flow of tests. The expected emotional flow can be refined based on various demographic data, contextual data (e.g., time of day, wait time for a proctor, and so forth), and other data. This data can vary over time, and users that traversed different overall paths may nonetheless share common edges on the graph. Thus, the expected emotional flow data can provide a rich source of information that can be used for improved process design, to weight user experience against other design designs, to gain business insights, and so forth.

In some embodiments, based on the expected emotional flow, parameters can be tuned for a given user's individual predictive emotional model based on deviations from expectations. For example, a highly emotive user that otherwise follows an expected flow can have a scale adjustment applied to any feedback received from the user. Similarly, a grumpy user can have an offset applied. By comparing individual users to expectations, outliers, such as the unusually cheerful, someone having a bad day, etc., can be better detected. In some embodiments, flags may be provided for a proctor's exit interview that indicate various issues such as the user running late, technical issues, and so forth, which may be considered when analyzing user behavior.

In some embodiments, emotional anomalies can be detected in the signals from the sentiment engine when comparing a user's personal predictive model to the expected emotional flow. Based on the anomalies, in some embodiments, a system can make decisions to steer the user through the outcome landscape toward more desirable paths, can adjust internal data on proctors and/or test design, and so forth. For example, if a user was in a bad mood when they started a test, their feedback regarding a proctor and/or the test procedure may be weighted or adjusted. In some embodiments, in addition to or instead of altering the user's flow through the outcome landscape, the sentiment engine can be used to tune parameters of artificial intelligence (AI) portions of a testing session. For example, the system can speed up or slow down a computerized voice speed, can change the amount of detail or guidance provided in an augmented reality (AR) application, and otherwise customize the user's testing experience.

In some embodiments, the systems and methods described above can be used to develop a dynamic, responsive map that reflects how users feel when navigating various test steps given various emotional contexts. This information can be used to highlight areas for improvement, steer business decisions, and so forth.

In some embodiments, the systems and methods described above can be used to examine the impact of A/B testing choices. For example, the systems and methods described herein can be used to determine the impact at both the subsystem and system level. For example, a user might have been happier with a faster preflight experience (e.g., introductory instructions and/or guidance), but the faster preflight may result in greater frustration during the testing session when the user encounters a more complex step that they struggle to understand and/or complete.

In some embodiments, the systems and methods described herein can be used with an AI model to steer patients through different test paths to maximize user happiness or other parameters, such as maximizing the likelihood of obtaining a valid result. For example, a user that is unhappy at a particular step of a test might be connected with a nicer, more experienced, and/or better-trained proctor. Other modifications to the testing experience can also be made, such as shortening or lengthening instructions, speeding up or slowing down a voice, and so forth.

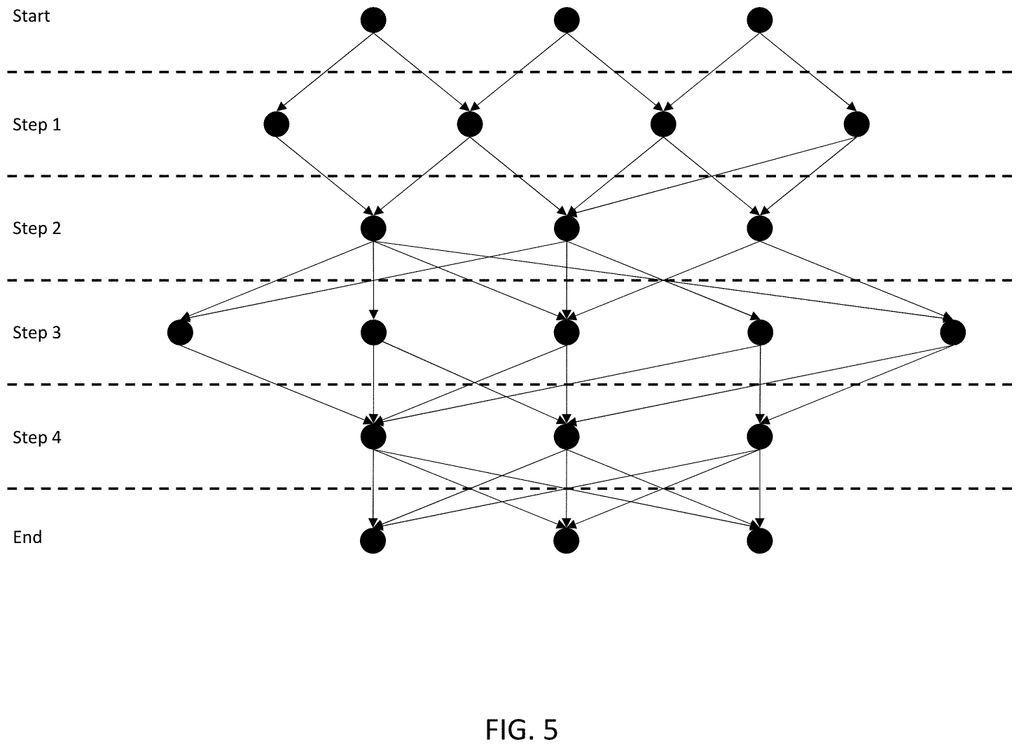

FIG. 5 shows an example outcome landscape according to some embodiments herein. The outcome landscape can be a directed graph comprising nodes and edges. In some embodiments, each node can represent a possible state that the user is in. For example, there can be one or more nodes at each step in a testing process, and at each node, a decision can be made as to which node to go to for the next step. As shown in FIG. 5, there can be several starting nodes. For example, users may be randomly assigned to one of several possible initial testing states, or a user may select an initial testing state. For example, a user may opt for an augmented reality experience, a text-based experience, etc. In some embodiments, a user may be directed to a particular experience if they are a new user (e.g., an experience that offers more guidance) and to a different test path if they have experience (for example, experience with the testing platform in general, experience with the particular test, and so forth), in which case the user may be provided with a more streamlined experience. In some embodiments, an individual user's path through the outcome graph can be updated as the user moves from node to node (e.g., from step to step), for example based at least in part on analysis of the user's sentiment (e.g., if the user appears frustrated, bored, angry, etc.). In some embodiments, users can be randomly assigned to a node at a particular step, for example for A/B testing or other research purposes. In some embodiments, the possible nodes at a next step can be determined at least in part by the node a user is on at a current step. That is, not all nodes at one step are necessarily connected to all nodes at the next step, although in some cases all nodes at one step may be connected to all nodes at a next step. Users can traverse the graph, moving from step to step and node to node, and eventually reaching an end state. In some embodiments, there can be multiple end states. In a typical testing example, there could be three possible end states, such as positive, negative, or indeterminate. In some embodiments, there can be additional end states. For example, a positive test result could be coupled with a recommendation to speak to a doctor via the platform, to seek medical attention, to obtain a prescription medication, to take a non-prescription medication, to monitor symptoms, and so forth. In some embodiments, an end state may recommend additional testing in the future (for example, for ongoing monitoring, to replace an inconclusive result, etc.). In some embodiments, the testing platform may compare the end states of testing sessions with a likelihood of returning to the platform for future testing.

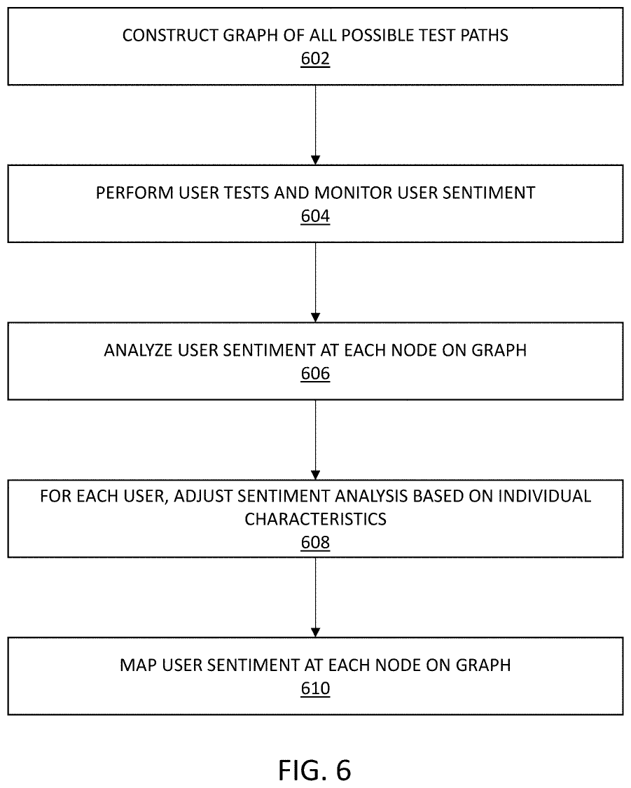

FIG. 6 shows an example process for measuring user experiences according to some embodiments. The example process of FIG. 6 can be run on a computing system. In some embodiments, steps can be performed in a different order, and/or there may be more or fewer steps than illustrated in FIG. 6. At 602, a system can be configured to construct a graph of all possible test paths. At 604, the system can perform user testing and monitor user sentiment during testing procedures. At 606, the system can analyze user sentiment at each visited node on the graph. In some embodiments, the system may, additionally or alternatively, generate an overall sentiment score that reflects whether the user had an overall positive or negative testing experience. At 608, the system can, for each user, adjust the sentiment analysis based on individual characteristics. For example, as discussed above, the system can adjust the sentiment analysis output based on, for example, whether the user was unusually upbeat, in a bad mood, in a rush, and so forth. In some embodiments, scores may be modified to account for particularities of the user and/or the testing session. Alternatively or additionally, in some embodiments, outlier testing sessions can be discarded or otherwise excluded, or given less weight than other testing sessions. In some embodiments, outlier sessions may be considered alone, for example to optimize testing flows for users who are in a rush, users who appear frustrated at the outset of the testing session, and so forth. At 610, the system can map user sentiment at each node on the graph. The information can be used for a variety of purposes. For example, a testing provider can identify particular nodes, paths through the graph, etc., that users find difficult, frustrating, or otherwise dislike. Based on this information, the testing provider can optimize the test flow to reduce the likelihood that a user will have a negative experience. Testing paths can be optimized overall (e.g., for all users) and/or for subsets of users, who may have different needs and/or different testing preferences.

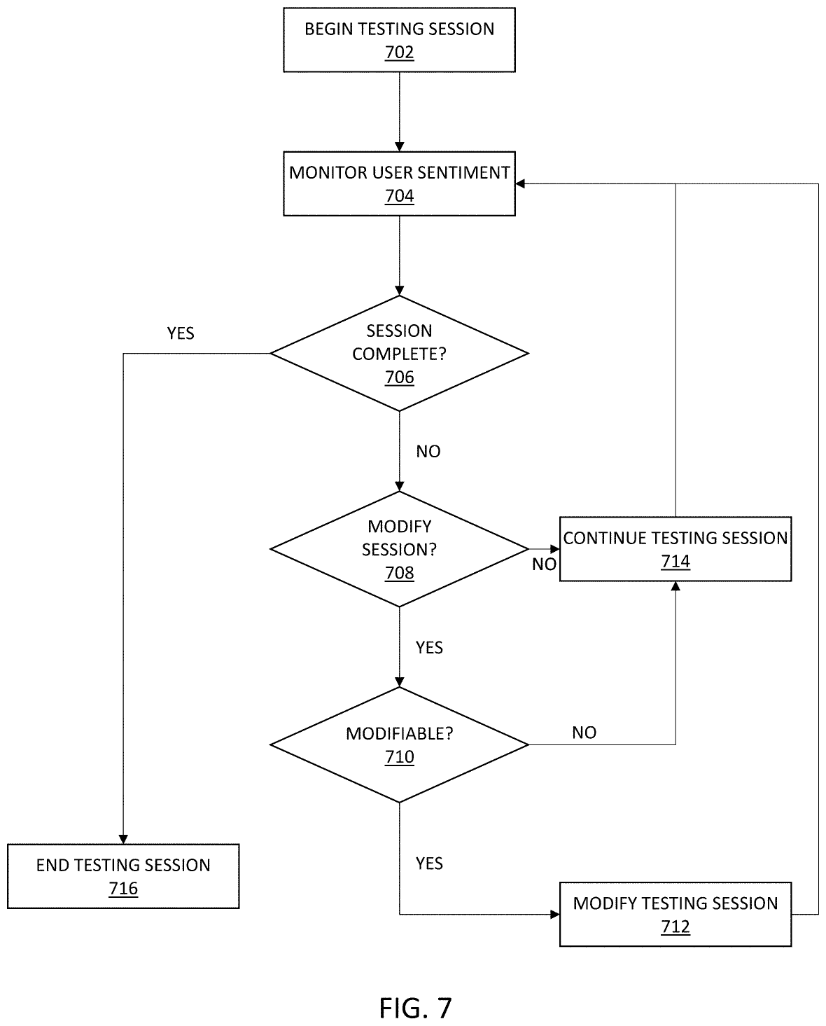

FIG. 7 shows an example test flow according to some embodiments. The example process of FIG. 7 can be run on a computing system. In some embodiments, steps can be performed in a different order, and/or there may be more or fewer steps than illustrated in FIG. 7. At 702, a user can begin a testing session. At 704, the system can monitor the user's sentiment. The system can monitor the user's sentiment continuously throughout the testing session, at fixed intervals during the testing session, at random points throughout the testing session, and so forth. At 706, the system can determine if the testing session has been substantially completed (e.g., the user has completed all the steps and is ready to receive results or has already received results). If the session is not complete, the system, at 708, can evaluate, based on the monitored sentiment, whether the user's test session should be modified (e.g., more guidance, less guidance, faster, slower, etc.). If the system determines that the user's session should be modified, the system can, at 710, determine if a modified session is available (for example, if there is more than one node at the next step and/or if there is a better node than a default node for the user). If the system determines that the user's session should not be modified or cannot be modified, the system can, at 714, continue the testing session and continue to monitor the testing session. If the system determines that the user's session should be modified and that there is a suitable modification available, the system can modify the testing session at 712, and the system can continue monitoring the user's sentiment. If, at 706, the testing session is substantially complete, the system can end the testing session at 716. In some embodiments, ending the testing session may include directing the user to further resources related to the testing session (e.g., information about treatment, medication, etc.), providing the user with a survey, providing a link to test results, and so forth.

### Computer Systems

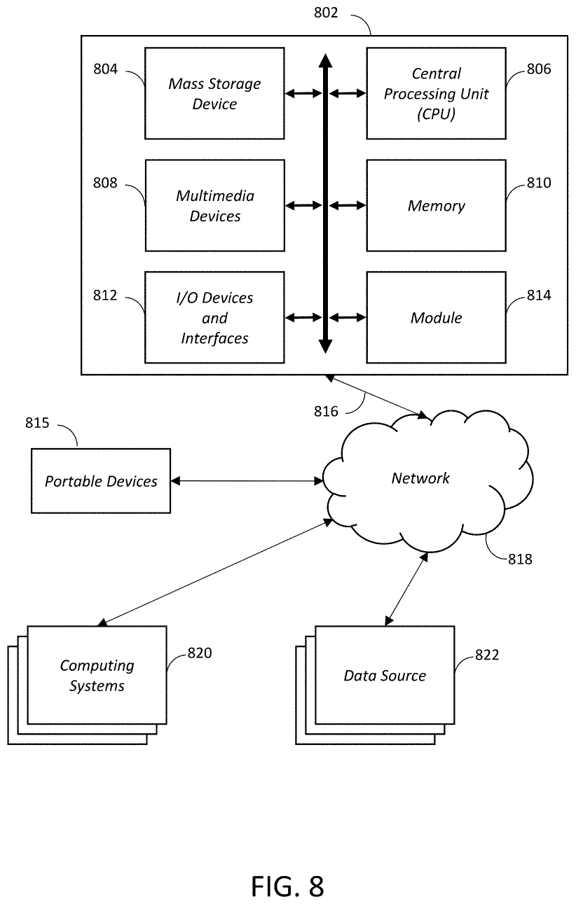

FIG. 8 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 8. The example computer system 802 is in communication with one or more computing systems 820 and/or one or more data sources 822 via one or more networks 818. While FIG. 8 illustrates an embodiment of a computing system 802, it is recognized that the functionality provided for in the components and modules of computer system 802 may be combined into fewer components and modules, or further separated into additional components and modules.

The computer system 802 can comprise a module 814 that carries out the functions, methods, acts, and/or processes described herein. The module 814 is executed on the computer system 802 by a central processing unit 806 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 802 includes one or more processing units (CPU) 806, which may comprise a microprocessor. The computer system 802 further includes a physical memory 810, such as random-access memory (RAM) for temporary storage of information, a read only memory (ROM) for permanent storage of information, and a mass storage device 804, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 802 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 802 includes one or more input/output (I/O) devices and interfaces 812, such as a keyboard, mouse, touch pad, and printer. The I/O devices and interfaces 812 can include one or more display devices, such as a monitor, that allows the visual presentation of data to a user. More particularly, a display device provides for the presentation of GUIs as application software data, and multi-media presentations, for example. The I/O devices and interfaces 812 can also provide a communications interface to various external devices. The computer system 802 may comprise one or more multi-media devices 808, such as speakers, video cards, graphics accelerators, and microphones, for example.

The computer system 802 may run on a variety of computing devices, such as a server, a Windows server, a Structure Query Language server, a Unix Server, a personal computer, a laptop computer, and so forth. In other embodiments, the computer system 802 may run on a cluster computer system, a mainframe computer system and/or other computing system suitable for controlling and/or communicating with large databases, performing high volume transaction processing, and generating reports from large databases. The computing system 802 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 802 illustrated in FIG. 8 is coupled to a network 818, such as a LAN, WAN, or the Internet via a communication link 816 (wired, wireless, or a combination thereof). Network 818 communicates with various computing devices and/or other electronic devices. Network 818 is communicating with one or more computing systems 820 and one or more data sources 822. The module 814 may access or may be accessed by computing systems 820 and/or data sources 822 through a web-enabled user access point. Connections may be a direct physical connection, a virtual connection, and other connection type. The web-enabled user access point may comprise a browser module that uses text, graphics, audio, video, and other media to present data and to allow interaction with data via the network 818.

Access to the module 814 of the computer system 802 by computing systems 820 and/or by data sources 822 may be through a web-enabled user access point such as the computing systems' 820 or data source's 822 personal computer, cellular phone, smartphone, laptop, tablet computer, e-reader device, audio player, or another device capable of connecting to the network 818. Such a device may have a browser module that is implemented as a module that uses text, graphics, audio, video, and other media to present data and to allow interaction with data via the network 818.

The output module may be implemented as a combination of an all-points addressable display such as a cathode ray tube (CRT), a liquid crystal display (LCD), a plasma display, or other types and/or combinations of displays. The output module may be implemented to communicate with input devices 812 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 802 may comprise a physical or logical connection established between a remote microprocessor and a mainframe host computer for the express purpose of uploading, downloading, or viewing interactive data and databases online in real-time. The remote microprocessor may be operated by an entity operating the computer system 802, including the client server systems or the main server system, an/or may be operated by one or more of the data sources 822 and/or one or more of the computing systems 820. In some embodiments, terminal emulation software may be used on the microprocessor for participating in the micro-mainframe link.

In some embodiments, computing systems 820 who are internal to an entity operating the computer system 802 may access the module 814 internally as an application or process run by the CPU 806.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a web site and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 802 may include one or more internal and/or external data sources (for example, data sources 822). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Cache), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 802 may also access one or more databases 822. The databases 822 may be stored in a database or data repository. The computer system 802 may access the one or more databases 822 through a network 818 or may directly access the database or data repository through I/O devices and interfaces 812. The data repository storing the one or more databases 822 may reside within the computer system 802.

### ADDITIONAL EMBODIMENTS

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A method for remote diagnostic testing comprising:
receiving, by a computer system from a user, a request to begin a testing session;
selecting, by the computer system, at least one guidance provision scheme from a plurality of guidance provision schemes;
beginning, by the computer system, the testing session using the selected at least one guidance provision scheme;
receiving, by the computer system, data indicative of one or more characteristics of the testing session;
determining, by the computer system based on the received data, to modify the testing session for the user; and
in response to determining to modify the testing session for the user, altering, by the computer system, the testing session.

2. The method of claim 1, wherein selecting the at least one guidance provision scheme is based on a user profile and a resource availability level.

3. The method of claim 2, wherein the user profile comprises at least one or a user experience level, demographic information, a number of times the user has taken a test, and information about previous positive or negative experiences of the user.

4. The method of claim 1, wherein receiving data indicative of one or more characteristics of the testing session comprises receiving data indicative of a user sentiment of the user, wherein determining to modify the testing session is based on the user sentiment.

5. The method of claim 4, further comprising:
determining, by the computer system based on the data indicative of the user sentiment, one or more baseline scores associated with one or more emotions; and
detecting, by the computer system, a change in the user sentiment during the testing session.

6. The method of claim 5, wherein determining to modify the testing session is based at least in part on detecting a change over a threshold amount of at least one of a negative emotion score, one or more baseline sentiment scores, or an overall sentiment score.

7. The method of claim 6, further comprising triggering one or more interventions, the one of more interventions comprising at least one of placing the user in a high priority queue, allocating the user a high-value resource, or modifying the testing session.

8. The method of claim 6, furthering comprising modifying a threshold amount based on a likelihood of a negative test outcome.

9. The method of claim 5, further comprising:
monitoring, by the computer system, user behavior, the user behavior comprising one or more of speech of the user, facial expressions of the user, and movements of the user, wherein the user data comprises data indicative of the user sentiment.

10. The method of claim 1, further comprising:
receiving, by the computer system from the user, a request for an adjustment to the testing session;
determining, by the computer system, that an adjustment to the testing session is available; and
modifying the testing session in response to the user request for an adjustment to the testing session.

11. The method of claim 10, further comprising:
determining by the computer system, a type of adjustment requested by the user,
wherein determining that an adjustment to the testing session is available comprises determining that an adjustment corresponding to the type of adjustment requested by the user is available.

12. A system for remote diagnostic testing comprising:
a non-transitory computer-readable medium with instructions encoded thereon; and
one or more processors configured to execute the instructions to cause the system to:
receive a request to begin a testing session from a user;
select at least one guidance provision scheme from a plurality of guidance provision schemes;
begin the testing session using the selected at least one guidance provision scheme;
receive data indicative of one or more characteristics of the testing session;
determine, based on the received data, to modify the testing session for the user; and
in response to determining to modify the testing session for the user, alter the testing session.

13. The system of claim 12, wherein selecting the at least one guidance provision scheme is based on a user profile and a resource availability level.

14. The system of claim 12, wherein receiving data indicative of one or more characteristics of the testing session comprises receiving data indicative of a user sentiment of the user, wherein determining to modify the testing session is based on the user sentiment.

15. The system of claim 14, wherein the instructions, when executed by the one or more processors, further cause the system to:
determine, based on the data indicative of the user sentiment, one or more baseline scores associated with one or more emotions; and
detect a change in the user sentiment during the testing session.

16. The system of claim 15, wherein determining to modify the testing session is based at least in part on detecting a change over a threshold amount of at least one of a negative emotion score, one or more baseline sentiment scores, or an overall sentiment score.

17. The system of claim 16, wherein the instructions, when executed by the one or more processors, further cause the system to trigger one or more interventions, the one of more interventions comprising at least one of placing the user in a high priority queue, allocating the user a high-value resource, or modifying the testing session.

18. The system of claim 15, wherein the instructions, when executed by the one or more processors, further cause the system to:
monitor user behavior, the user behavior comprising one or more of speech of the user, facial expressions of the user, and movements of the user, wherein the user data comprises data indicative of the user sentiment.

19. The system of claim 12, wherein the instructions, when executed by the one or more processors, further cause the system to:
receive, from the user, a request for an adjustment to the testing session;
determine that an adjustment to the testing session is available; and
modify the testing session in response to the user request for an adjustment to the testing session.

20. The system of claim 19, wherein the instructions, when executed by the one or more processors, further cause the system to:
determine a type of adjustment requested by the user,
wherein determining that an adjustment to the testing session is available comprises determining that an adjustment corresponding to the type of adjustment requested by the user is available.
